(19) United States
(12) Patent Application Publication
(10) Pub. No.: US 2023/0310027 A1
(43) Pub. Date: Oct. 5, 2023

(54) METHODS AND DEVICES FOR TROCAR PLACEMENT

(71) Applicant: BIOTEX, INC., Houston, TX (US)

(72) Inventors: Kimberly Neely, Houston, TX (US); Charles Houssiere, Houston, TX (US); Ashok Gowda, Bellaire, TX (US)

(21) Appl. No.: 18/012,976

(22) PCT Filed: Jun. 28, 2021
(86) PCT No.: PCT/US2021/039469

§ 371 Date: Dec. 26, 2022

Related U.S. Application Data
(60) Provisional application No. 63/045,139, filed on Jun. 28, 2020.

(51) Int. Cl. A61B 17/34; A61B 17/02

(52) U.S. Cl. CPC A61B 17/3462; A61B 17/0218; A61B 2017/348

(57) ABSTRACT

A method and device for placing and/or retaining medical devices on an animal or subject is provided that may include an anchoring device having a body with a channel or aperture through which a medical device may pass and be reversibly retained at given degrees of insertion, a retaining mechanism for controlling the release or retention of the medical device in the channel or aperture, and an attachment feature for attaching the anchoring device to an animal or subject (e.g. onto the skin of a human). The anchoring device may be attached to a subject, such as over the site of an incision or other point of access to the interior of the subject, and a medical device may be inserted into the interior via the channel or aperture in the body of the anchoring device and retained by action of the retention mechanism.

### CROSS-REFERENCE TO RELATED APPLICATIONS

This Patent Cooperation Treaty international patent application claims the benefit and priority of U.S. Provisional Patent Application Ser. No. 63/045,139, filed Jun. 28, 2020, entitled “METHODS AND DEVICES FOR TROCAR PLACEMENT”, the contents of all of the foregoing applications are hereby incorporated by reference in their entireties.

### FIELD OF THE INVENTION

This invention relates to methods and devices for placing and/or retaining medical devices on an animal or subject, particularly to placing and/or retaining trocars and related devices on an animal or subject, and more particularly to placing and/or retaining trocars and related devices on an animal or subject with an attached anchoring device.

### BACKGROUND OF THE INVENTION

A trocar (or trochar) is a medical or veterinary device that is made up of an obturator (which may be a metal or plastic sharpened or non-bladed tip), a cannula (essentially a hollow tube), and a seal. Trocars are placed through the abdomen during laparoscopic surgery. The trocar functions as a portal for the subsequent placement of other instruments, such as graspers, scissors, staplers, etc. Trocars also allow the escape of gas or fluid from organs within the body. Trocars are used in medicine to access and drain collections of fluid such as in a patient with hydrothorax or ascites. Surgical trocars are used to perform laparoscopic (keyhole) surgery. They are deployed as a means of introduction for cameras and laparoscopic hand instruments, such as scissors, graspers, etc., to perform surgery hitherto carried out by making a large abdominal incision (“open” surgery), something that has revolutionized patient care.

When surgeons use the Hasson technique for trocar placement, they incise the skin. Other trocar placement techniques do not require an initial incision, at least not a large one. The Hasson incision is large enough that gas can escape between the flesh and the cannula. Surgeons prevent this by 1) using a cone-shaped anchor to plug the gaps or 2) using a purse-string suture or clamps to tighten the flesh around the cannula. Because cone-shaped anchors plug the wound under tension, they can come out of the body, bringing the cannulas with them. Surgeons suture the anchor to the body to mitigate this effect, but they spend extra time doing so.

### SUMMARY OF THE INVENTION

This invention relates to methods and devices for placing and/or retaining medical devices on an animal or subject, particularly to placing and/or retaining trocars and related devices on an animal or subject, and more particularly to placing and/or retaining trocars and related devices on an animal or subject with an attached anchoring device.

In general, a method and device for placing and/or retaining medical devices on an animal or subject (which may be referred to in general as the subject or the like herein) may include an anchoring device having a body with a channel or aperture through which a medical device may pass and be reversibly retained at given degrees of insertion, a retaining mechanism for controlling the release or retention of the medical device in the channel or aperture, and an attachment feature for attaching the anchoring device to an animal or subject (e.g. onto the skin of a human). The anchoring device may be attached to a subject, such as over the site of an incision or other point of access to the interior of the subject, and a medical device may be inserted into the interior via the channel or aperture in the body of the anchoring device and retained by action of the retention mechanism.

In a first aspect of the invention, an anchoring device for placing and/or retaining medical devices on an animal or subject includes a body with at least a partially cylindrical or tube-like portion through which a channel or aperture is present to provide access to the interior of a subject from the outside. In any of the embodiments of the invention, the body may generally be formed from a flexible and/or elastic material such that it may be reversibly deformed to alter the size of the channel or aperture to allow a medical device to be inserted, such as by widening or expanding the channel or aperture, and then retaining the medical device, such as by releasing the widening or expanding force such that the channel or aperture reverts to a smaller size to press and/or hold onto the inserted medical device. The flexible and/or elastic material may also facilitate bending and/or deforming of the body of the anchoring device such that the position or angle of insertion of the medical device into the interior of the subject may be changed. The flexible and/or elastic material may further facilitate forming a seal against the inserted medical device, such as to prevent fluid and/or gas leakage from the interior of the subject, or to prevent external substances from entering.

In a second aspect of the invention, the anchoring device may utilize a retention mechanism to alter the size or shape of the channel or aperture in the body and to provide a retention action on an inserted medical device in the channel or aperture. In any of the embodiments of the invention, the retention mechanism may generally constrict or compress the channel or aperture of the body of the anchoring device to provide a retaining force on an inserted medical device. The retention mechanism may utilize the compression action of the material of the channel or aperture (e.g. the inserted medical device is larger than the channel or aperture such that the material stretches to allow passage of the medical device and thus retains it by compression on the medical device due to the elastic tension of the material). The retention mechanism may also include other features to create or increase the retaining force or action on the inserted medical device, such as, for example, a spring(s), clip(s), and/or any other appropriate feature that may act to constrict or compress the channel or aperture about the inserted medical device to retain it.

In a third aspect of the invention, the anchoring device may utilize an actuating mechanism to control the retaining function of the retention mechanism, as discussed above. In any of the embodiments of the invention, the actuating mechanism may generally allow a user to alternate the retention mechanism between a retaining configuration, where the channel or aperture of the body acts on the inserted medical device to hold it in place, and a release or insertion configuration, where the channel or aperture of the body is widened, deformed or otherwise configured to allow the medical device to be inserted, repositioned or moved (i.e. the retaining force or action of the channel or aperture is reduced to enable free or less restricted movement of the medical device in the channel or aperture). Actuating mechanisms may include, for example, handles, tabs, switches, levers and/or any other appropriate actuator which may be utilized to affect the retention mechanism, such as by acting directly on the channel or aperture to widen or constrict it, or by affecting a feature that creates or increases the retaining force or action of the channel or aperture.

In a fourth aspect of the invention, the anchoring device may include at least one portion for attaching or affixing the anchoring device to a subject. In any of the embodiments of the invention, the anchoring device may include an attachment feature for attaching the anchoring device to an animal or subject (e.g. onto the skin of a human). The anchoring device may be attached to a subject, such as over the site of an incision or other point of access to the interior of the subject. The attachment feature may, in general adhere and/or conform to the surface of the subject around the point of access, such as the skin or other tissue, to attach the anchoring device to the subject and/or form a seal against fluid, gas or other substances from passing from outside of the anchoring device into the interior of the subject or vice versa. The attachment feature may include an attachment surface which may be of a flexible or deformable material and/or configuration such that the attachment surface may conform to the surface of the subject. The attachment surface may further include an adhesive and/or a sealant to adhere and/or seal the attachment surface to the surface of the subject. The attachment feature may also include a portion that may insert into the incision or other point of access to the interior of the subject to attach the anchoring device to the subject, such as by friction fitting, retaining ridges or ribs, and/or any other appropriate method of fixing to the incision or point of access. For example, the body of the anchoring device may include sections of alternating width or diameter such that it may be inserted into an incision or point of access until a section is able to resist removal of the anchoring device.

The present invention together with the above and other advantages may best be understood from the following detailed description of the embodiments of the invention and as illustrated in the drawings. The following description, while indicating various embodiments of the invention and numerous specific details thereof, is given by way of illustration and not of limitation. Many substitutions, modifications, additions or rearrangements may be made within the scope of the invention, and the invention includes all such substitutions, modifications, additions or rearrangements.

### BRIEF DESCRIPTION OF THE FIGURES

FIG. 1 illustrates a side view of an embodiment of an anchoring device with an inserted medical device of the present invention;

FIG. 1a illustrates a cross-sectional view of an anchoring device;

FIG. 1B illustrates a top view of an anchoring device;

FIGS. 2-2o illustrate embodiments of an anchoring device with different actuating mechanisms and handles;

FIGS. 3-3c illustrate placement of an anchoring device and medical device on a subject;

FIGS. 4-4c illustrate embodiments of an anchoring device with conforming and/or deforming enhancing features;

FIGS. 5 and 5a illustrate embodiments of an anchoring device with retaining tabs and/or additional sealing features;

FIGS. 6-6c illustrate embodiments of an anchoring device with cone-like portions and/or retaining ribs;

FIGS. 7 and 7a illustrate embodiments of an anchoring device with handling features;

FIGS. 7b and 7c illustrate full away film usage on the attachment feature of an anchoring device; and

FIGS. 8, 8a, 8b, 8c, 8d, 8e and 8f illustrate use of an anchoring device for tissue manipulation.

### DETAILED DESCRIPTION OF THE INVENTION

The detailed description set forth below is intended as a description of the presently exemplified systems, devices and methods provided in accordance with aspects of the present invention and are not intended to represent the only forms in which the present invention may be prepared or utilized. It is to be understood, rather, that the same or equivalent functions and components may be accomplished by different embodiments that are also intended to be encompassed within the spirit and scope of the invention. Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the exemplary methods, devices and materials are now described. All publications mentioned herein are incorporated herein by reference for the purpose of describing and disclosing, for example, the designs and methodologies that are described in the publications which might be used in connection with the presently described invention. The publications listed or discussed above, below and throughout the text are provided solely for their disclosure prior to the filing date of the present application. Nothing herein is to be construed as an admission that the inventors are not entitled to antedate such disclosure by virtue of prior invention.

This invention relates to methods and devices for placing and/or retaining medical devices on an animal or subject, particularly to placing and/or retaining trocars and related devices on an animal or subject, and more particularly to placing and/or retaining trocars and related devices on an animal or subject with an attached anchoring device.

In general, a method and device for placing and/or retaining medical devices on an animal or subject (which may be referred to in general as the subject or the like herein) may include an anchoring device having a body with a channel or aperture through which a medical device may pass and be reversibly retained at given degrees of insertion, a retaining mechanism for controlling the release or retention of the medical device in the channel or aperture, and an attachment feature for attaching the anchoring device to an animal or subject (e.g. onto the skin of a human). The anchoring device may be attached to a subject, such as over the site of an incision or other point of access to the interior of the subject, and a medical device may be inserted into the interior via the channel or aperture in the body of the anchoring device and retained by action of the retention mechanism.

The medical device may generally be any device or implement that may be inserted into a subject, such as a trocar or portions thereof, cannula, needle, probe, tube, sensing device, and/or any other appropriate device.

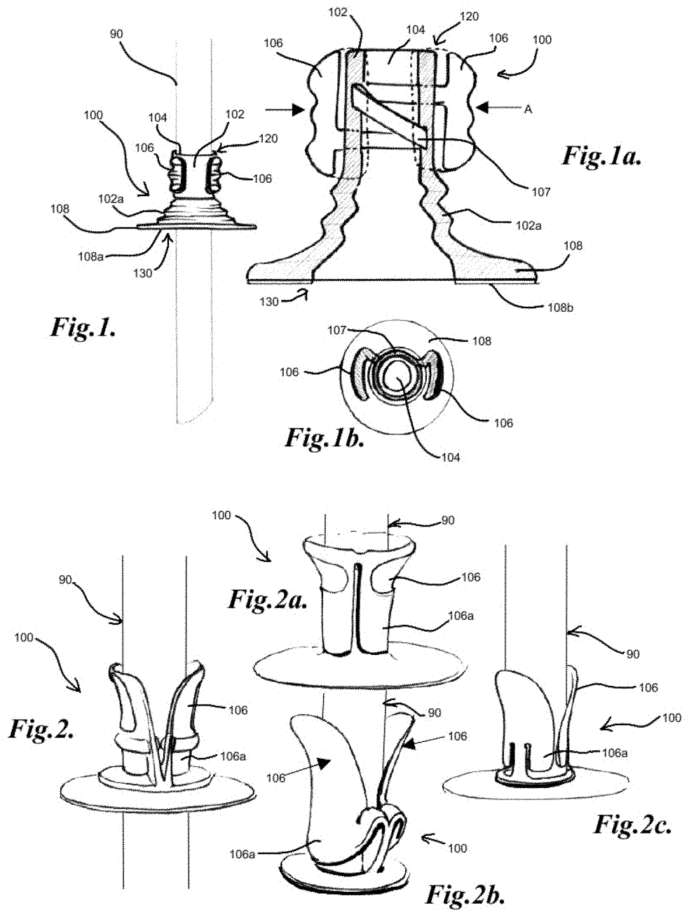

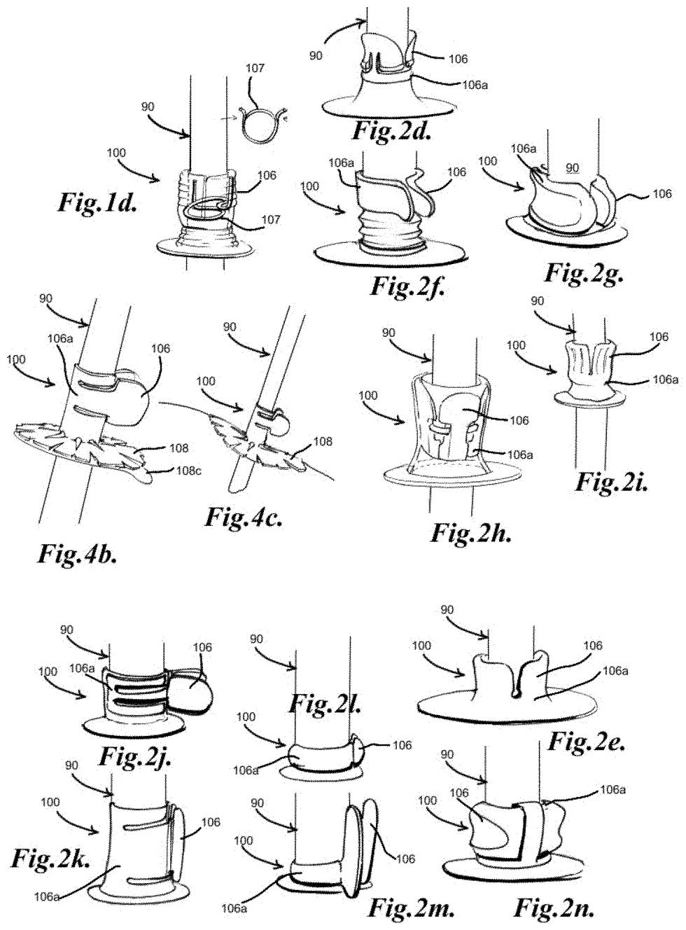

In a first aspect of the invention, an anchoring device, such as the anchoring device 100 illustrated in the side view of FIG. 1 and the top view of FIG. 1B, for placing and/or retaining medical devices on an animal or subject includes a body with at least a partially cylindrical or tube-like portion through which a channel or aperture is present to provide access to the interior of a subject from the outside. FIG. 1 illustrates an example of an anchoring device 100 with a body 102, shown as a generally cylindrical or tube-like body with a channel 104 passing from the top end 120 (which faces out from the subject) to the bottom end 130 (which faces the point of access to the interior of the subject).

In any of the embodiments of the invention, the body 102 may generally be formed from a flexible and/or elastic material such that it may be reversibly deformed to alter the size of the channel or aperture to allow a medical device to be inserted, such as by widening or expanding the channel or aperture, and then retaining the medical device, such as by releasing the widening or expanding force such that the channel or aperture reverts to a smaller size to press and/or hold onto the inserted medical device, as shown with the channel 104 retaining the medical device 90. The flexible and/or elastic material may also facilitate bending and/or deforming of the body of the anchoring device such that the position or angle of insertion of the medical device 90 into the interior of the subject may be changed. The flexible and/or elastic material may further facilitate forming a seal against the inserted medical device 90, such as to prevent fluid and/or gas leakage from the interior of the subject, or to prevent external substances from entering.

In any of the embodiments of the invention, the material of the body 102 may include natural or synthetic polymeric elastomers, such as silicone, natural rubber, synthetic rubber (e.g. styrene butadiene, polyisoprene, polychloroprene a.k.a. neoprene, etc.), and/or any other appropriate elastomer or combination thereof. In general, the material may be selected for biocompatibility or non-reactivity in medical settings, ability to be sterilized (e.g. by autoclave, ethylene oxide, ultraviolet, etc.).

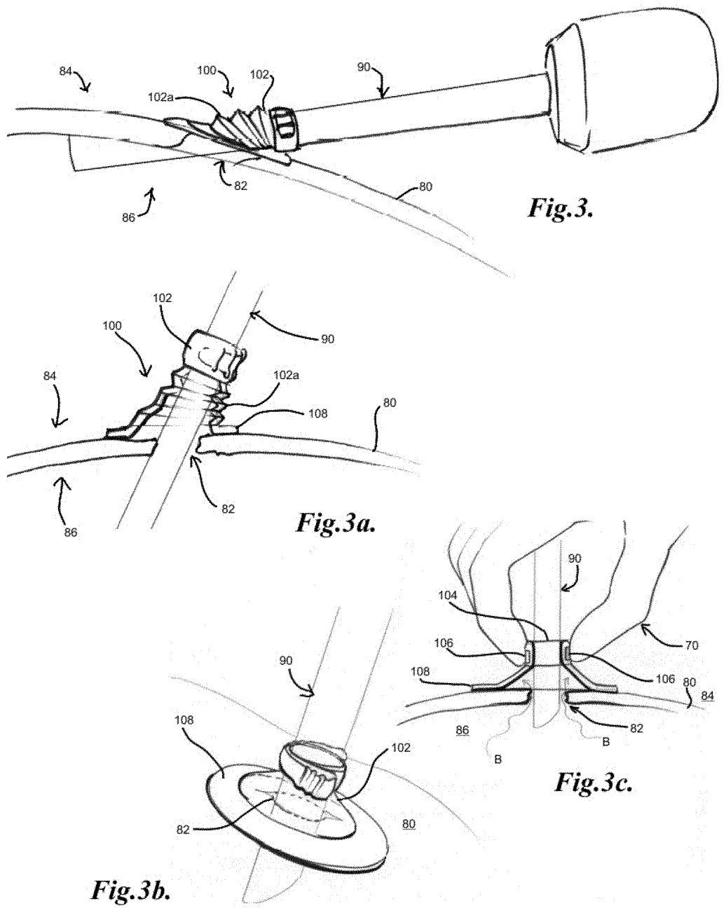

In any of the embodiments of the invention, the body 102 may also include a portion that is adapted to flex and/or deform, such as the flexing portion 102a. The flexing portion 102a may, for example, include pleats, folds or other formations, as illustrated with pleats in FIGS. 1, la, 3 and 3a. This may be utilized to, for example, change the angle of insertion of the medical device 90, as shown in FIGS. 3 and 3a.

In any of the embodiments of the invention, the channel 104 may also widen in regions, such as towards the bottom end 130, as shown in the cross-sectional view of FIG. 1a. This may be desirable to cover a larger area on the subject, such as to completely cover the incision or other point of access on the subject, as shown with the access point 82 on the subject 80 (e.g. skin of an animal or person) from the outside 84 to the interior 86, as shown with the partial see-through view of FIG. 3b. The widened portion may also be utilized for angling the inserted medical device 90, as shown in FIGS. 3 and 3a with the insertion of the medical device 90 at an angle, the flexing of the flexing portion 102a and being accommodated by the widened channel 104.

In a second aspect of the invention, the anchoring device, such as the anchoring device 100, may utilize a retention mechanism to alter the size or shape of the channel or aperture (e.g. channel 104) in the body 102 and to provide a retention action on an inserted medical device 90 in the channel or aperture (e.g. channel 104). In any of the embodiments of the invention, the retention mechanism may generally constrict or compress the channel or aperture of the body of the anchoring device to provide a retaining force on an inserted medical device. The retention mechanism may utilize the compression action of the material of the channel or aperture (e.g. the inserted medical device is larger than the channel or aperture such that the material stretches to allow passage of the medical device and thus retains it by compression on the medical device due to the elastic tension of the material).

In any of the embodiments of the invention, the retention mechanism may also include other features to create or increase the retaining force or action on the inserted medical device, such as, for example, a spring(s), clip(s), and/or any other appropriate feature that may act to constrict or compress the channel or aperture about the inserted medical device to retain it. FIG. 1a illustrates a spring which presses inward on the channel 104 to retain an inserted medical device, shown as a torsion spring 107 in the material of the body 102 and around the channel 104. Other examples of springs may also be utilized, such as helical springs, conical springs, leaf springs, and/or any other appropriate springs. FIGS. 5 and 5a illustrate a tab 109 which may bias against the inserted medical device 90 for retaining in the channel 104. The channel 104 may further include additional sealing features, such as the membrane(s) 105 in FIGS. 5 and 5a to aid in sealing against the medical device 90 where the main body 102 does not constrict against the medical device 90.

In a third aspect of the invention, the anchoring device, such as the anchoring device 100, may utilize an actuating mechanism to control the retaining function of the retention mechanism, as discussed above. In any of the embodiments of the invention, the actuating mechanism may generally allow a user to alternate the retention mechanism between a retaining configuration, where the channel or aperture of the body acts on the inserted medical device to hold it in place, and a release or insertion configuration, where the channel or aperture of the body is widened, deformed or otherwise configured to allow the medical device to be inserted, repositioned or moved (i.e. the retaining force or action of the channel or aperture is reduced to enable free or less restricted movement of the medical device in the channel or aperture). Actuating mechanisms may include, for example, handles, tabs, switches, levers and/or any other appropriate actuator which may be utilized to affect the retention mechanism, such as by acting directly on the channel or aperture to widen or constrict it, or by affecting a feature that creates or increases the retaining force or action of the channel or aperture. FIGS. 1, 1a, 1b, 1d, 3b, 3c, 7 and 7a illustrate an actuating mechanism as handles 106 which may be pressed or pinched by a user 70 to affect the retention mechanism, such as to expand the torsion spring 107 in FIGS. 1a, 1d, 6 and 6a to release or loosen the hold on the inserted medical device 90 or to open the channel 104 wider to allow insertion of the medical device 90. In general, the actuating mechanism may be acted on by the user 70 to widen or deform the channel 104, followed by insertion of the medical device 90 into the channel 104 to the desired degree, and then the user 70 may release the actuating mechanism to cause the channel 104 or other feature to retain the medical device 90 at the desired degree of insertion.

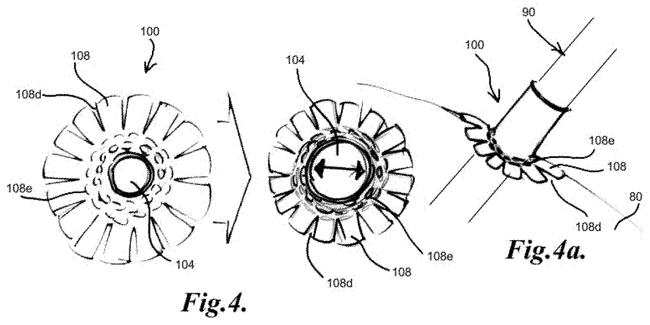

In any of the embodiments of the invention, the actuating mechanism may form part of or act as the retention mechanism. FIGS. 2-2o and 4b-4c illustrate different examples of handles 106 as part of retaining portions 106a attached to portions of the body 102 which constrict the channel 104 (e.g. acting as a leaf spring or the like, or in a vertical compression C to release/loosen the retaining portion 106a similar to a Tuohy-Borst adapter in FIG. 2o) and release/loosen the channel 104 when the handles 106 are pressed to deform the retaining portions 106a. The different shaped handles 106 may also be utilized with a spring 107, such as illustrated in FIGS. 1d, 6 and 6a. The body 102 of the anchoring device 100 may also include features to aid the actuation mechanism in widening or deforming the channel 104, such as with perforations, slits or other areas of the material of the body 102 removed to make deformation easier, as illustrated with the perforations 108e in FIGS. 4 and 4a to aid in widening the channel 104.

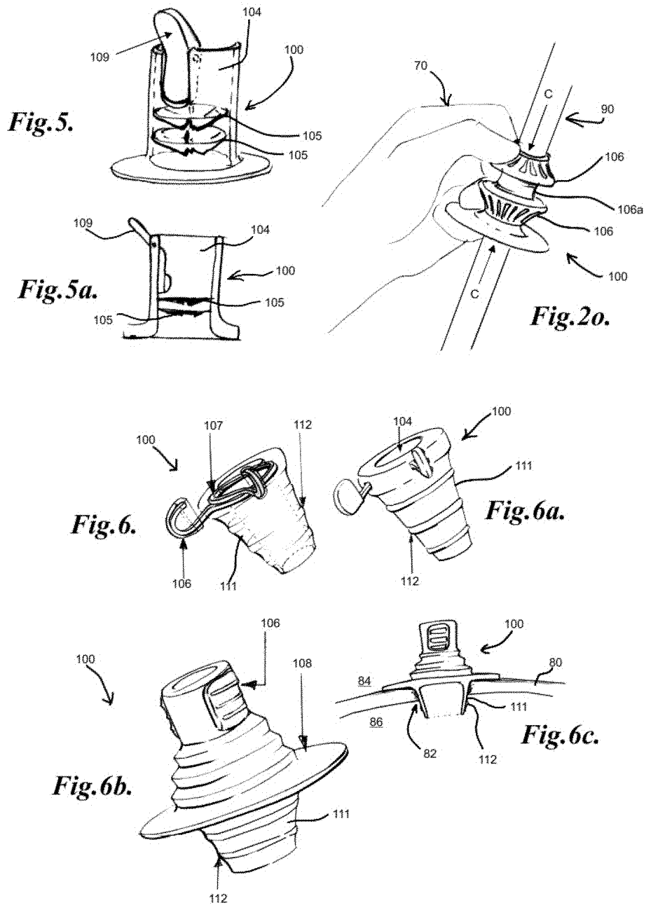

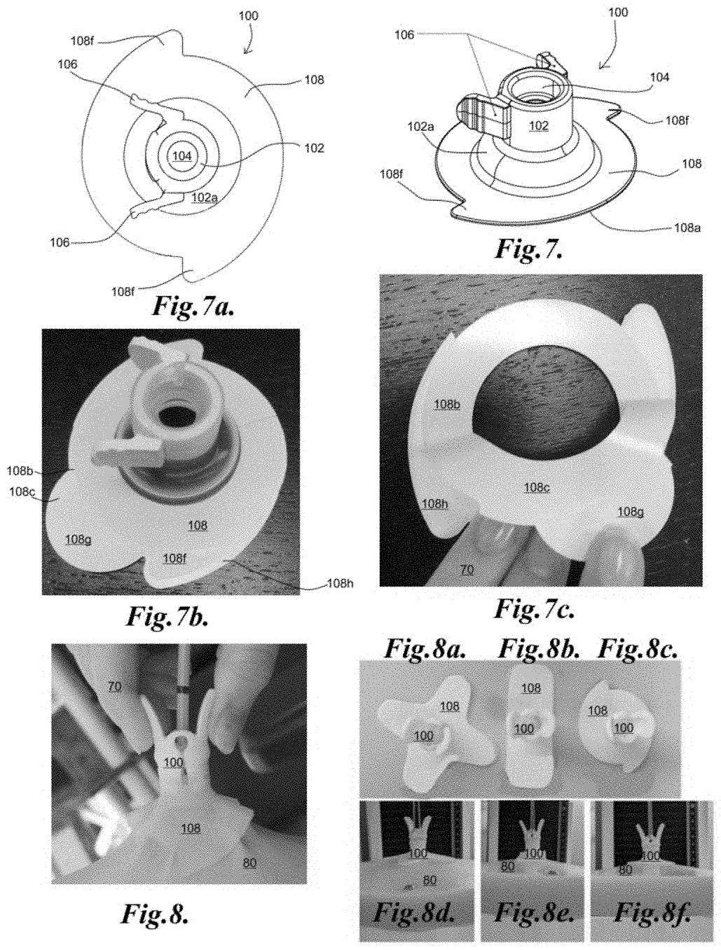

In a fourth aspect of the invention, the anchoring device, such as the anchoring device 100, may include at least one portion for attaching or affixing the anchoring device to a subject. In any of the embodiments of the invention, the anchoring device 100 may include an attachment feature for attaching the anchoring device to an animal or subject (e.g. onto the skin of a human), such as the attachment features 108 illustrated in any of the figures. The anchoring device 100 may be attached to a subject 80, such as over the site of an incision or other point of access to the interior of the subject, as illustrated with the access point 82 on the subject 80 in FIGS. 3-3c. The attachment feature 108, as illustrated in the figures as a flattened disc-like portion, may, in general adhere and/or conform to the surface of the subject 80 around the point of access 82, such as the skin or other tissue, to attach the anchoring device 100 to the subject 80 and/or form a seal against fluid, gas or other substances from passing from outside 84 of the anchoring device 100 into the interior of the subject 86 or vice versa, such as, for example, as illustrated with fluids B in FIG. 3c. The attachment feature 108 may include an attachment surface 108a, such as illustrated in FIG. 1, which may be of a flexible or deformable material and/or configuration such that the attachment surface 108a may conform to the surface of the subject 80. The configuration may also include features for enhancing the conformation, such as slits, scores, cuts, feathering or other changes to the shape of the attachment feature 108. FIGS. 4, 4a, 4b and 4c illustrate an anchoring device 100 with an attachment feature 108 with slits 108d to form a feathered or flowering configuration such that the attachment feature 108 may deform more than an unperturbed edge to conform better to the subject 80. The attachment surface 108a may further include an adhesive and/or a sealant to adhere and/or seal the attachment surface 108a to the surface of the subject 80, as shown with adhesive pad 108b in FIG. 1a. The adhesive or sealant may further be covered with a protective film or other removable material to prevent adhesion before use, as shown with the pull away film 108c covering the adhesive pad 108b in FIG. 4b, which may be removed prior to placement on the subject 80. The pull away film 108c may further include a handling feature, such as, for example, a protruding portion 108g to aid in gripping by a user for removing the pull away film 108c from the adhesive pad 108b, as illustrated with FIGS. 4b, 7b and 7c. The pull away film 108c may also include portions that are not removed, such as, for example, portions 108h in FIGS. 7b and 7c. The attachment feature 108 may also include a portion that may insert into the incision or other point of access to the interior of the subject to attach the anchoring device 100 to the subject 80, such as by friction fitting, retaining ridges or ribs, and/or any other appropriate method of fixing to the incision or point of access. For example, the body of the anchoring device may include sections of alternating width or diameter such that it may be inserted into an incision or point of access until a section is able to resist removal of the anchoring device, as illustrated with the cone-like portion 111 with ribs 112 which may retain themselves after pressing past the point of access 82 which is slightly smaller than the appropriate rib 112 in FIGS. 6, 6a, 6b and 6c.

In some embodiments, the attachment feature 108 may include features for aiding in removal of the anchoring device 100 from a surface, such as the surface of the subject 80 (e.g. skin, or other tissue). The features may include, for example, portions that aid in gripping of the attachment feature 108 by a user, such as variations in the outline of the attachment feature 108 to, for example, provide areas that are more easily handled to begin pealing off the attachment feature 108 from a surface. FIGS. 7, 7a, 7b and 7c illustrate handling portions 108f which protrude from the main area of the attachment feature 108. The handling portions 108f may also have an adhesive pad 108b or other attachment feature to adhere to a surface. In some embodiments, the adhesive pad 108b on the handling portions 108f may be covered by the pull away film 108c and may also include portions 108h that remain such that the handling portions 108f do not become adhered to the surface to, for example, enable easier grasping and handling for removal of the attachment feature 108 from the surface.

In one aspect, the anchoring device 100 may be utilized to raise or “tent” a subject's 80 tissue, for example, in preparation for laparoscopic surgery to raise the skin/tissue of a subject 80. FIGS. 8-8f illustrate the usage of an anchoring device 100 to raise the skin/tissue of a subject 80 by pulling on the anchoring device 100 by a user 70 once attached (e.g. via the attachment feature 108) to the surface of the subject 80. FIG. 8 illustrates the lifting by a user 70 pulling on the anchoring device 100 after it is attached (e.g. via adhesion of attachment feature 108) to a surface of a subject 80. FIGS. 8a, 8b and 8c illustrate different shapes of attachment features 108 for raising or “tenting” a subject's 80 tissue by pulling after attachment. FIG. 8a illustrates a cross-shaped attachment feature (with the corresponding example of pulling of tissue shown in FIG. 8d), FIG. 8b illustrates a rectangular-shaped attachment feature (with the corresponding example of pulling of tissue shown in FIG. 8e), and FIG. 8c illustrates a round-shaped attachment feature (with the corresponding example of pulling of tissue shown in FIG. 8f). The raised or “tented” tissue of a subject 80 may then be situated with the anchoring device 100 for insertion of a medical device, such as through a channel 104, or in uses without using the anchoring device 100 for an access point.

Although the invention has been described with respect to specific embodiments thereof, these embodiments are merely illustrative, and not restrictive of the invention. The description herein of illustrated embodiments of the invention, including the description in the Abstract and Summary, is not intended to be exhaustive or to limit the invention to the precise forms disclosed herein. Rather, the description is intended to describe illustrative embodiments, features and functions in order to provide a person of ordinary skill in the art context to understand the invention without limiting the invention to any particularly described embodiment, feature or function, including any such embodiment feature or function described in the Abstract or Summary. While specific embodiments of, and examples for, the invention are described herein for illustrative purposes only, various equivalent modifications are possible within the spirit and scope of the invention, as those skilled in the relevant art will recognize and appreciate. As indicated, these modifications may be made to the invention in light of the foregoing description of illustrated embodiments of the invention and are to be included within the spirit and scope of the invention. Thus, while the invention has been described herein with reference to particular embodiments thereof, a latitude of modification, various changes and substitutions are intended in the foregoing disclosures, and it will be appreciated that in some instances some features of embodiments of the invention will be employed without a corresponding use of other features without departing from the scope and spirit of the invention as set forth. Therefore, many modifications may be made to adapt a particular situation or material to the essential scope and spirit of the invention.

Reference throughout this specification to “one embodiment”, “an embodiment”, or “a specific embodiment” or similar terminology means that a particular feature, structure, or characteristic described in connection with the embodiment is included in at least one embodiment and may not necessarily be present in all embodiments. Thus, respective appearances of the phrases “in one embodiment”, “in an embodiment”, or “in a specific embodiment” or similar terminology in various places throughout this specification are not necessarily referring to the same embodiment. Furthermore, the particular features, structures, or characteristics of any particular embodiment may be combined in any suitable manner with one or more other embodiments. It is to be understood that other variations and modifications of the embodiments described and illustrated herein are possible in light of the teachings herein and are to be considered as part of the spirit and scope of the invention.

In the description herein, numerous specific details are provided, such as examples of components and/or methods, to provide a thorough understanding of embodiments of the invention. One skilled in the relevant art will recognize, however, that an embodiment may be able to be practiced without one or more of the specific details, or with other apparatus, systems, assemblies, methods, components, materials, parts, and/or the like. In other instances, well-known structures, components, systems, materials, or operations are not specifically shown or described in detail to avoid obscuring aspects of embodiments of the invention. While the invention may be illustrated by using a particular embodiment, this is not and does not limit the invention to any particular embodiment and a person of ordinary skill in the art will recognize that additional embodiments are readily understandable and are a part of this invention.

As used herein, the terms “comprises,” “comprising,” “includes,” “including,” “has,” “having,” or any other variation thereof, are intended to cover a non-exclusive inclusion. For example, a process, product, article, or apparatus that comprises a list of elements is not necessarily limited only those elements but may include other elements not expressly listed or inherent to such process, process, article, or apparatus.

1. A device for retaining a medical device comprising:
a body having a substantially tube-like shape with a top end and a bottom end;
a channel through said body from said top end to said bottom end;
an attachment feature proximal to said bottom end having an attachment surface adapted to conform to a portion of an animal subject; and
an actuating mechanism adapted to alter the retention of an inserted medical device in said channel.

2. The device of claim 1, wherein said body further comprises a retention feature that compresses against said inserted medical device in said channel.

3. The device of claim 2, wherein said retention feature is selected from the group consisting of a tab, a spring and a compressing elastic portion.

4. The device of claim 2, wherein said actuating mechanism is adapted to release or loosen said retention feature.

5. The device of claim 2, wherein said actuating mechanism comprises at least a pair of handles adapted for pinching by a user to actuate said retention feature.

6. The device of claim 1, wherein said attachment feature comprises an adhering feature.

7. The device of claim 6, further comprising a pull away film adapted to cover at least a portion of said adhering feature to prevent adhesion prior to removal of said pull away film.

8. The device of claim 7, wherein a portion of said pull away film is adapted to remain on said adhering feature.

9. The device of claim 1, wherein said attachment feature comprises at least one handling feature adapted to aid in removal of said device from a surface.

10. The device of claim 9, wherein said at least one handling feature comprises a protrusion of said attachment feature.

11. The device of claim 1, wherein said body comprises a flexing portion adapted to adjust the angle of said channel.

12. A device for retaining attaching to a surface of an animal subject comprising:
a body having a substantially tube-like shape with a top end and a bottom end;
an attachment feature proximal to said bottom end having an attachment surface adapted to conform to a portion of an animal subject; and
a handling feature adapted for grasping by a user to pull said device for deformation of said attachment surface.
